(19) United States
(12) Patent Application Publication
(10) Pub. No.: US 2002/0107369 A1
(43) Pub. Date: Aug. 8, 2002

(54) Growth differentiation factor-10

(76) Inventors: Se-Jin Lee, Baltimore, MD (US); Noreen Cunningham, Silver Spring, MD (US); John Cunningham, Siver Spring, MD (US)

Correspondence Address: GARY CARY WARE & FRIENDENRICH LLP, 4365 EXECUTIVE DRIVE, SUITE 1600, SAN DIEGO, CA, 92121-2189, US

(21) Appl. No.: 09/813,459

(22) Filed: Mar. 20, 2001

Related U.S. Application Data
(63) Continuation of application No. 08/624,635, filed on Oct. 10, 1996.
Application No. 08/624,635 is a 371 of international application No. PCT/US94/11440, filed on Oct. 7, 1994.
Application No. 08/624,635 is a 371 of international application No. 08/134,078, filed on Oct. 8, 1993.

(51) Int. Cl. C07K 16/00; C12P 21/08

(52) U.S. Cl. 530/388.24

(57) ABSTRACT

Growth differentiation factor-10 (GDF-10) is disclosed along with its polynucleotide and amino acid sequence. Also disclosed are diagnostic and therapeutic methods of using the GDF-10 polypeptide and polynucleotide sequences.

### BACKGROUND OF THE INVENTION

1. Field of the Invention

The invention relates generally to growth factors and specifically to a new member of the transforming growth factor beta (TGF-β) super-family, which is denoted, growth differentiation factor-10 (GDF-10).

2. Description of Related Art

The transforming growth factor β (TGF-β) superfamily encompasses a group of structurally-related proteins which affect a wide range of differentiation processes during embryonic development. The family includes, Mullerian inhibiting substance (MIS), which is required for normal male sex development (Behringer, et al., Nature, 345:167, 1990), Drosophila decapentaplegic (DPP) gene product, which is required for dorsal-ventral axis formation and morphogenesis of the imaginal disks (Padgett, et al., Nature, 325:81-84, 1987), the Xenopus Vg-1 gene product, which localizes to the vegetal pole of eggs ((Weeks, et al., Cell 51:861-867, 1987), the activins (Mason, et al., Biochem, Biophys. Res. Commun., 135:957-964, 1986), which can induce the formation of mesoderm and anterior structures in Xenopus embryos (Thomsen, et al., Cell, 63:485, 1990), and the bone morphogenetic proteins (BMPs, osteogenin, OP-1) which can induce de novo cartilage and bone formation (Sampath, et al., J. Biol. Chem., 265:13198, 1990). The TGF-βs can influence a variety of differentiation processes, including adipogenesis, myogenesis, chondrogenesis, hematopoiesis, and epithelial cell differentiation (for review, see Massague, Cell 49:437, 1987).

The proteins of the TGF-β family are initially synthesized as a large precursor protein which subsequently undergoes proteolytic cleavage at a cluster of basic residues approximately 110-140 amino acids from the C-terminus. The C-terminal regions, or mature regions, of the proteins are all structurally related and the different family members can be classified into distinct subgroups based on the extent of their homology. Although the homologies within particular subgroups range from 70% to 90% amino acid sequence identity, the homologies between subgroups are significantly lower, generally ranging from only 20% to 50%. In each case, the active species appears to be a disulfide-linked dimer of C-terminal fragments. For most of the family members that have been studied, the homodimeric species has been found to be biologically active, but for other family members, like the inhibins (Ling, et al., Nature, 321:779, 1986) and the TGF-βs (Cheifetz, et al., Cell, 48:409, 1987), heterodimers have also been detected, and these appear to have different biological properties than the respective homodimers.

Identification of new factors that are tissue-specific in their expression pattern will provide a greater understanding of that tissue's development and function and allow development of effective diagnostic and therapeutic regimens.

### SUMMARY OF THE INVENTION

The present invention provides a cell growth and differentiation factor, GDF-10, a polynucleotide sequence which encodes the factor, and antibodies which are immunoreactive with the factor. This factor appears to relate to various cell proliferative disorders, especially those involving those involving uterine, nerve, bone, and adipose tissue.

Thus, in one embodiment, the invention provides a method for detecting a cell proliferative disorder of uterine, nerve, or fat origin and which is associated with GDF-10. In another embodiment, the invention provides a method for treating a cell proliferative disorder by suppressing or enhancing GDF-10 activity.

### BRIEF DESCRIPTION OF THE DRAWINGS

FIG. 1 shows expression of GDF-10 mRNA in adult tissues.

FIG. 2 shows nucleotide and predicted amino acid sequence murine GDF-10. Consensus N-glycosylation signals are denoted by plain boxes.

FIG. 3 shows the alignment of the C-terminal sequences of GDF-10 with other members of the TGF-β superfamily. The conserved cysteine residues are boxed. Dashes denote gaps introduced in order to maximize alignment.

FIG. 4 shows amino acid homologies with different members of the TGF-β superfamily. Numbers represent percent amino acid identities between each pair calculated from the first conserved cysteine to the C-terminus.

FIG. 5 shows an alignment of the C-terminal sequences of human (top lines) and murine (bottom lines) GDF-10.

FIG. 6 shows an autoradiogram of labeled secreted proteins synthesized by 293 cells transfected with a pcDNAI vector into which the GDF-10 cDNA was inserted in either the antisense (lanes 1 and 2) or sense (lanes 3 and 4) orientation.

### DETAILED DESCRIPTION OF THE INVENTION

The present invention provides a growth and differentiation factor, GDF-10 and a polynucleotide sequence encoding GDF-10. GDF-10 is expressed at highest levels in uterus and fat and at lower levels in other tissues, such as brain. In one embodiment, the invention provides a method for detection of a cell proliferative disorder of uterine, nerve, or fat origin which is associated with GDF-10 expression. In another embodiment, the invention provides a method for treating a cell proliferative disorder by using an agent which suppresses or enhances GDF-10 activity.

The TGF-β superfamily consists of multifunctional polypeptides that control proliferation, differentiation, and other functions in many cell types. Many of the peptides have regulatory, both positive and negative, effects on other peptide growth factors. The structural homology between the GDF-10 protein of this invention and the members of the TGF-β family, indicates that GDF-10 is a new member of the family of growth and differentiation factors. Based on the known activities of many of the other members, it can be expected that GDF-10 will also possess biological activities that will make it useful as a diagnostic and therapeutic reagent.

The expression of GDF-10 in uterine and fat tissue suggests a variety of applications using the polypeptide, polynucleotide, and antibodies of the invention, related to contraception, fertility, pregnancy, and cell proliferative diseases. Abnormally low levels of the factor my be indicative of impaired function in the uterus while abnormally high levels may be indicative of hypertrophy, hyperplasia, or the presence of ectopic tissue. Hence, GDF-10 my be useful in detecting not only primary and metastatic neoplasms of uterine origin but in detecting diseases such as endometriosis as well. In addition, GDF-10 may also be useful as an indicator of developmental anomalies in prenatal screening procedures.

Several members of the TGF-β superfamily possess activities suggesting possible applications for the treatment of cell proliferative disorders, such as cancer. In particular, TGF-β has been shown to be potent growth inhibitor for a variety of cell types (Massague, Cell 49:437, 1987). MIS has been shown to inhibit the growth of human endometrial carcinoma tumors in nude mice (Donahoe, et al., Ann. Surg. 194:472, 1981), and inhibin a has been shown to suppress the development of tumors both in the ovary and in the testis (Matzuk, et al., Nature, 360:313, 1992). GDF-10 may have similar activity and may therefore be useful as an anti-proliferative agent, such as for the treatment of endometrial cancer or endometriosis.

Many of the members of the TGF-β family are also important mediators of tissue repair. TGF-β has been shown to have marked effects on the formation of collagen and causes of striking angiogenic response in the newborn mouse (Roberts, et al., Proc. Nat'l Acad. Sci., USA 83:4167, 1986). The BMP's can induce new bone growth and are effective for the treatment of fractures and other skeletal defects (Glowacki, et al., Lancet, 1:959, 1981; Ferguson, et al., Clin. Orthoped. Relat Res., 227:265, 1988; Johnson, et al., Clin Orthoped Relat. Res., 230:257, 1988). Based on the high degree of homology between GDF-10 and BMP-3, GDF-10 may have similar activities and may be useful in repair of tissue injury caused by trauma or burns for example.

GDF-10 may play a role in regulation of the menstrual cycle or regulation of uterine function during pregnancy, and therefore, GDF-10, anti-GDF-10 antibodies, or antisense polynucleotides may be useful either in contraceptive regimens, in enhancing the success of in vitro fertilization procedures, or in preventing premature labor.

Certain members of this superfamily have expression patterns or possess activities that relate to the function of the nervous system. For example, one family member, namely GDNF, has been shown to be a potent neurotrophic factor that can promote the survival of dopaminergic neurons (Lin, et al., Science, 260:1130). Another family member, namely dorsalin, is capable of promoting the differentiation of neural crest cells (Baster, et al., Cell, 73:687). The inhibins and activins have been shown to be expressed in the brain (Meunier, et al., Proc. Nat'l Acad. Sci., USA, 85:247, 1988; Sawchenko, et al., Nature, 334:615, 1988), and activin has been shown to be capable of functioning as a nerve cell survival molecule (Schubert, et al., Nature, 344:868, 1990). Another family member, namely GDF-1, is nervous system-specific in its expression pattern (Lee, Proc. Nat'l Acad. Sci., USA, 88:4250, 1991), and certain other family members, such as Vgr-1 (Lyons, et al., Proc. Nat'l Acad. Sci., USA, 86:4554, 1989; Jones et al., Development, 111:581, 1991), OP-1 (Ozkaynak, et al., J. Biol. Chem., 267:25220, 1992), and BMP-4 (Jones, et al., Development, 111:531, 1991), are also known to be expressed in the nervous system. By analogy GDF-10 may have applications in the treatment of neurodegenerative diseases or in maintaining cells or tissues in culture prior to transplantation.

The expression of GDF-10 in adipose tissue also raises the possibility of applications for GDF-10 in the treatment of obesity or of disorders related to abnormal proliferation of adipocytes. In this regard, TGF-β has been shown to be a potent inhibitor of adipocyte differentiation in vitro (Ignotz and Massague, Proc. Natl. Acad. Sci., USA 82:8530,1985).

The term “substantially pure” as used herein refers to GDF-10 which is substantially free of other proteins, lipids, carbohydrates or other materials with which it is naturally associated. One skilled in the art can purify GDF-10 using standard techniques for protein purification. The substantially pure polypeptide will yield a single major band on a non-reducing polyacrylamide gel. The purity of the GDF-10 polypeptide can also be determined by amino-terminal amino acid sequence analysis. GDF-10 polypeptide includes functional fragments of the polypeptide, as long as the activity of GDF-10 remains. Smaller peptides containing the biological activity of GDF-10 are included in the invention.

The invention provides polynucleotides encoding the GDF-10 protein. These polynucleotides include DNA, cDNA and RNA sequences which encode GDF-10. It is understood that all polynucleotides encoding all or a portion of GDF-10 are also included herein, as long as they encode a polypeptide with GDF-10 activity. Such polynucleotides include naturally occurring, synthetic, and intentionally manipulated polynucleotides. For example, GDF-10 polynucleotide may be subjected to site-directed mutagenesis. The polynucleotide sequence for GDF-10 also includes antisense sequences. The polynucleotides of the invention include sequences that are degenerate as a result of the genetic code. There are 20 natural amino acids, most of which are specified by more than one codon. Therefore, all degenerate nucleotide sequences are included in the invention as long as the amino acid sequence of GDF-10 polypeptide encoded by the nucleotide sequence is functionally unchanged.

Specifically disclosed herein is a cDNA sequence for GDF-10 which is 2322 base pairs in length and contains an open reading frame beginning with a methionine codon at nucleotide 126. The encoded polypeptide is 476 amino acids in length with a molecular weight of about 52.5 kD, as determined by nucleotide sequence analysis. The GDF-10 sequence contains a core of hydrophobic amino acids near the N-terminus, suggestive of a signal sequence for secretion. GDF-10 contains four potential N-glycosylation sites at asparagine residues 114, 152, 277, and 467. GDF-10 contains several potential proteolytic processing sites. Cleavage most likely occurs following arginine 365, which would generate a mature fragment of GDF-10 predicted to be 111 amino acids in length and have an unglycosylated molecular weight of about 12.6 kD, as determined by nucleotide sequence analysis. One skilled in the art can modify, or partially or completely remove, the glycosyl groups from the GDF-10 protein using standard techniques. Therefore the functional protein or fragments thereof of the invention includes glycosylated, partially glycosylated and unglycosylated species of GDF-10.

The C-terminal region of GDF-10 following the putative proteolytic processing site shows significant homology to the known members of the TGF-β superfamily. The GDF-10 sequence contains most of the residues that are highly conserved in other family members. Among the known family mammalian TGF-β family members, GDF-10 is most homologous to BMP-3 (83% sequence identity beginning with the first conserved cysteine residue). GDF-10 also shows significant homology to BMP-3 (approximately 30% sequence identity) in the pro-region of the molecule. Based on these sequence comparisons, GDF-10 and BMP-3 appear to define a new subfamily within the larger superfamily.

Minor modifications of the recombinant GDF-10 primary amino acid sequence may result in proteins which have substantially equivalent activity as compared to the GDF-10 polypeptide described herein. Such modifications may be deliberate, as by site-directed mutagenesis, or may be spontaneous. All of the polypeptides produced by these modifications are included herein as long as the biological activity of GDF-10 still exists. Further, deletion of one or more amino acids can also result in a modification of the structure of the resultant molecule without significantly altering its biological activity. This can lead to the development of a smaller active molecule which would have broader utility. For example, one can remove amino or carboxy terminal amino acids which are not required for GDF-10 biological activity.

The nucleotide sequence encoding the GDF-10 polypeptide of the invention includes the disclosed sequence and conservative variations thereof. The term “conservative variation” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acid, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

DNA sequences of the invention can be obtained by several methods. For example, the DNA can be isolated using hybridization techniques which are well known in the art. These include, but are not limited to: 1) hybridization of genomic or cDNA libraries with probes to detect homologous nucleotide sequences, 2) polymerase chain reaction (PCR) on genomic DNA or cDNA using primers capable of annealing to the DNA sequence of interest, and 3) antibody screening of expression libraries to detect cloned DNA fragments with shared structural features.

Preferably the GDF-10 polynucleotide of the invention is derived from a mammalian organism, and most preferably from a mouse, rat, or human. Screening procedures which rely on nucleic acid hybridization make it possible to isolate any gene sequence from any organism, provided the appropriate probe is available. Oligonucleotide probes, which correspond to a part of the sequence encoding the protein in question, can be synthesized chemically. This requires that short, oligopeptide stretches of amino acid sequence must be known. The DNA sequence encoding the protein can be deduced from the genetic code, however, the degeneracy of the code must be taken into account. It is possible to perform a mixed addition reaction when the sequence is degenerate. This includes a heterogeneous mixture of denatured double-stranded DNA. For such screening, hybridization is preferably performed on either single-stranded DNA or denatured double-stranded DNA. Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA clone by the hybridization of the target DNA to that single probe in the mixture which is its complete complement (Wallace, et al., Nucl. Acid Res., 9:879, 1981).

The development of specific DNA sequences encoding GDF-10 can also be obtained by: 1) isolation of double-stranded DNA sequences from the genomic DNA; 2) chemical manufacture of a DNA sequence to provide the necessary codons for the polypeptide of interest; and 3) in vitro synthesis of a double-stranded DNA sequence by reverse transcription of mRNA isolated from a eukaryotic donor cell; In the latter case, a double-stranded DNA complement of mRNA is eventually formed which is generally referred to as cDNA.

Of the three above-noted methods for developing specific DNA sequences for use in recombinant procedures, the isolation of genomic DNA isolates is the least common. This is especially true when it is desirable to obtain the microbial expression of mammalian polypeptides due to the presence of introns.

The synthesis of DNA sequences is frequently the method of choice when the entire sequence of amino acid residues of the desired polypeptide product is known. When the entire sequence of amino acid residues of the desired polypeptide is not known, the direct synthesis of DNA sequences is not possible and the method of choice is the synthesis of cDNA sequences. Among the standard procedures for isolating cDNA sequences of interest is the formation of plasmid- or phage-carrying cDNA libraries which are derived from reverse transcription of mRNA which is abundant in donor cells that have a high level of genetic expression. When used in combination with polymerase chain reaction technology, even rare expression products can be cloned. In those cases where significant portions of the amino acid sequence of the polypeptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single-stranded form (Jay, et al., Nucl. Acid Res., 11:2325, 1983).

A cDNA expression library, such as lambda gt11, can be screened indirectly for GDF-10 peptides having at least one epitope, using antibodies specific for GDF-10. Such antibodies can be either polyclonally or monoclonally derived and used to detect expression product indicative of the presence of GDF-10 cDNA.

DNA sequences encoding GDF-10 can be expressed in vitro by DNA transfer into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

In the present invention, the GDF-10 polynucleotide sequences may be inserted into a recombinant expression vector. The term “recombinant expression vector” refers to a plasmid, virus or other vehicle known in the art that has been manipulated by insertion or incorporation of the GDF-10 genetic sequences. Such expression vectors contain a promoter sequence which facilitates the efficient transcription of the inserted genetic sequence of the host. The expression vector typically contains an origin of replication, a promoter, as well as specific genes which allow phenotypic selection of the transformed cells. Vectors suitable for use in the present invention include, but are not limited to the T7-based expression vector for expression in bacteria (Rosenberg, et al., Gene, 56:125, 1987), the pMSXND expression vector for expression in mammalian cells (Lee and Nathans, J. Biol. Chem., 263:3521, 1988) and baculovirus-derived vectors for expression in insect cells. The DNA segment can be present in the vector operably linked to regulatory elements, for example, a promoter (e.g., T7, metallothionein I, or polyhedrin promoters).

Polynucleotide sequences encoding GDF-10 can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art. Such vectors are used to incorporate DNA sequences of the invention. Preferably, the mature C-terminal region of GDF-10 is expressed from a cDNA clone containing the entire coding sequence of GDF-10. Alternatively, the C-terminal portion of GDF-10 can be expressed as a fusion protein with the pro-region of another member of the TGF-β family or co-expressed with another pro-region (see for example, Hammonds, et al., Molec. Endocrin. 5:149, 1991; Gray, A., and Mason, A., Science, 247:1328, 1990).

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be cotransformed with DNA sequences encoding the GDF-10 of the invention, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. (see for example, Eukaryotic Viral Vectors, Cold Spring Harbor Laboratory, Gluzman ed., 1982).

Isolation and purification of microbial expressed polypeptide, or fragments thereof, provided by the invention, may be carried out by conventional means including preparative chromatography and immunological separations involving monoclonal or polyclonal antibodies.

The invention includes antibodies immunoreactive with GDF-10 polypeptide or functional fragments thereof. Antibody which consists essentially of pooled monoclonal antibodies with different epitopic specificities, as well as distinct monoclonal antibody preparations are provided. Monoclonal antibodies are made from antigen containing fragments of the protein by methods well known to those skilled in the art (Kohler, et al., Nature, 256:495, 1975). The term antibody as used in this invention is meant to include intact molecules as well as fragments thereof, such as Fab and F(ab′)₂, which are capable of binding an epitopic determinant on GDF-10.

The term “cell-proliferative disorder” denotes malignant as well as non-malignant cell populations which often appear to differ from the surrounding tissue both morphologically and genotypically. The term “cell-proliferative disorder” also includes situations in which a normally occurring process could be enhanced or suppressed for clinical benefit; an example of such a process would be fracture healing. Malignant cells (i.e. cancer) develop as a result of a multistep process. The GDF-10 polynucleotide that is an antisense molecule is useful in treating malignancies of the various organ systems, particularly, for example, cells in uterine or adipose tissue. Essentially, any disorder which is etiologically linked to altered expression of GDF-10 could be considered susceptible to treatment with a GDF-10 suppressing reagent. One such disorder is a malignant cell proliferative disorder, for example.

The invention provides a method for detecting a cell proliferative disorder of uterine or adipose tissue which comprises contacting an anti-GDF-10 antibody with a cell suspected of having a GDF-10 associated disorder and detecting binding to the antibody. The antibody reactive with GDF-10 is labeled with a compound which allows detection of binding to GDF-10. For purposes of the invention, an antibody specific for GDF-10 polypeptide may be used to detect the level of GDF-10 in biological fluids and tissues. Any specimen containing a detectable amount of antigen can be used. A preferred sample of this invention is uterine or fat tissue. The level of GDF-10 in the suspect cell can be compared with the level in a normal cell to determine whether the subject has a GDF-10-associated cell proliferative disorder. Preferably the subject is human.

The antibodies of the invention can be used in any subject in which it is desirable to administer in vitro or in vivo immunodiagnosis or immunotherapy. The antibodies of the invention are suited for use, for example, in immunoassays in which they can be utilized in liquid phase or bound to a solid phase carrier. In addition, the antibodies in these immunoassays can be detectably labeled in various ways. Examples of types of immunoassays which can utilize antibodies of the invention are competitive and non-competitive immunoassays in either a direct or indirect format. Examples of such immunoassays are the radioimmunoassay (RIA) and the sandwich (immunometric) assay. Detection of the antigens using the antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

The antibodies of the invention can be bound to many different carriers and used to detect the presence of an antigen comprising the polypeptide of the invention. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

There are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds. Those of ordinary skill in the art will know of other suitable labels for binding to the antibody, or will be able to ascertain such, using routine experimentation.

Another technique which may also result in greater sensitivity consists of coupling the antibodies to low molecular weight haptens. These haptens can then be specifically detected by means of a second reaction. For example, it is common to use such haptens as biotin, which reacts with avidin, or dinitrophenyl, puridoxal, and fluorescein, which can react with specific antihapten antibodies.

In using the monoclonal antibodies of the invention for the in vivo detection of antigen, the detectably labeled antibody is given a dose which is diagnostically effective. The term “diagnostically effective” means that the amount of detectably labeled monoclonal antibody is administered in sufficient quantity to enable detection of the site having the antigen comprising a polypeptide of the invention for which the monoclonal antibodies are specific.

The concentration of detectably labeled monoclonal antibody which is administered should be sufficient such that the binding to those cells having the polypeptide is detectable compared to the background. Further, it is desirable that the detectably labeled monoclonal antibody be rapidly cleared from the circulatory system in order to give the best target-to-background signal ratio.

As a rule, the dosage of detectably labeled monoclonal antibody for in vivo diagnosis will vary depending on such factors as age, sex, and extent of disease of the individual. Such dosages may vary, for example, depending on whether multiple injections are given, antigenic burden, and other factors known to those of skill in the art.

For in vivo diagnostic imaging, the type of detection instrument available is a major factor in selecting a given radioisotope. The radioisotope chosen must have a type of decay which is detectable for a given type of instrument. Still another important factor in selecting a radioisotope for in vivo diagnosis is that deleterious radiation with respect to the host is minimized. Ideally, a radioisotope used for in vivo imaging will lack a particle emission, but produce a large number of photons in the 140-250 keV range, which may readily be detected by conventional gamma cameras.

For in vivo diagnosis radioisotopes may be bound to immunoglobulin either directly or indirectly by using an intermediate functional group. Intermediate functional groups which often are used to bind radioisotopes which exist as metallic ions to immunoglobulins are the bifunctional chelating agents such as diethylenetriaminepentacetic acid (DTPA) and ethylenediaminetetraacetic acid (EDTA) and similar molecules. Typical examples of metallic ions which can be bound to the monoclonal antibodies of the invention are ¹¹¹In, ⁹⁷Ru, ⁶⁷Ga, ⁶⁸Ga, ⁷²As, ⁸⁹Zr, and ²⁰¹Tl.

The monoclonal antibodies of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements which are particularly useful in such techniques include ¹⁵⁷Gd, ⁵⁵Mn, ¹⁶²Dy, ⁵²Cr, and ⁵⁶Fe.

The monoclonal antibodies of the invention can be used in vitro and in vivo to monitor the course of amelioration of a GDF-10-associated disease in a subject. Thus, for example, by measuring the increase or decrease in the number of cells expressing antigen comprising a polypeptide of the invention or changes in the concentration of such antigen present in various body fluids, it would be possible to determine whether a particular therapeutic regimen aimed at ameliorating the GDF-10-associated disease is effective. The term “ameliorate” denotes a lessening of the detrimental effect of the GDF-10-associated disease in the subject receiving therapy.

The present invention identifies a nucleotide sequence that can be expressed in an altered manner as compared to expression in a normal cell, therefore it is possible to design appropriate therapeutic or diagnostic techniques directed to this sequence. Thus, where a cell-proliferative disorder is associated with the expression of GDF-10, nucleic acid sequences that interfere with GDF-10 expression at the translational level can be used. This approach utilizes, for example, antisense nucleic acid and ribozymes to block translation of a specific GDF-10 mRNA, either by masking that mRNA with an antisense nucleic acid or by cleaving it with a ribozyme.

Antisense nucleic acids are DNA or RNA molecules that are complementary to at least a portion of a specific mRNA molecule (Weintraub, Scientific American, 262:40, 1990). In the cell, the antisense nucleic acids hybridize to the corresponding mRNA, forming a double-stranded molecule. The antisense nucleic acids interfere with the translation of the mRNA, since the cell will not translate a mRNA that is double-stranded. Antisense oligomers of about 15 nucleotides are preferred, since they are easily synthesized and are less likely to cause problems than larger molecules when introduced into the target GDF-10-producing cell. The use of antisense methods to inhibit the in vitro translation of genes is well known in the art (Marcus-Sakura, Anal. Biochem., 172:289, 1988).

Ribozymes are RNA molecules possessing the ability to specifically cleave other single-stranded RNA in a manner analogous to DNA restriction endonucleases. Through the modification of nucleotide sequences which encode these RNAs, it is possible to engineer molecules that recognize specific nucleotide sequences in an RNA molecule and cleave it (Cech, J.Amer.Med. Assn., 260:3030, 1988). A major advantage of this approach is that, because they are sequence-specific, only mRNAs with particular sequences are inactivated.

There are two basic types of ribozymes namely, tetrahymena-type (Hasselhoff, Nature, 334:585, 1988) and “hammerhead”-type. Tetrahymena-type ribozymes recognize sequences which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to tetrahymena-type ribozymes for inactivating a specific mRNA species and 18-based recognition sequences are preferable to shorter recognition sequences.

The present invention also provides gene therapy for the treatment of cell proliferative disorders which are mediated by GDF-10 protein. Such therapy would achieve its therapeutic effect by introduction of the GDF-10 antisense polynucleotide into cells having the proliferative disorder. Delivery of antisense GDF-10 polynucleotide can be achieved using a recombinant expression vector such as a chimeric virus or a colloidal dispersion system. Especially preferred for therapeutic delivery of antisense sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. By inserting a GDF-10 sequence of interest into the viral vector, along with another gene which encodes the ligand for a receptor on a specific target cell, for example, the vector is now target specific. Retroviral vectors can be made target specific by inserting, for example, a polynucleotide encoding a sugar, a glycolipid, or a protein. Preferred targeting is accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector containing the GDF-10 antisense polynucleotide.

Since recombinant retroviruses are defective, they require assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence which enables the packaging mechanism to recognize an RNA transcript for encapsidation. Helper cell lines which have deletions of the packaging signal include, but are not limited to Ψ2, PA317 and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

Alternatively, NIH 3T3 or other tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for GDF-10 antisense polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77,1981). In addition to mammalian cells, liposomes have been used for delivery of polynucleotides in plant, yeast and bacterial cells. In order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

Due to the expression of GDF-10 primarily in uterine and adipose tissue, there are a variety of applications using the polypeptide, polynucleotide, and antibodies of the invention, related to these and other tissues. Such applications include treatment of cell proliferative disorders involving these and other tissues, including bone. In addition, GDF-10 may be useful in various gene therapy procedures.

The following examples are intended to illustrate but not limit the invention. While they are typical of those that might be used, other procedures known to those skilled in the art may alternatively be used.

### EXAMPLE 1

### Identification and Isolation of a Novel TGF-β Family Member

To identify new members of the TGF-β superfamily, degenerate oligonucleotides were designed which corresponded to two conserved regions among the known family members: one region downstream of the first conserved cysteine residue and the other region spanning the invariant cysteine residues near the C-terminus. These primers were used for polymerase chain reactions on lung and brain cDNA followed by subcloning the PCR products using restriction sites placed at the 5′ ends of the primers, picking individual E. coli colonies carrying these subcloned inserts, and using a combination of random sequencing and hybridization analysis to eliminate know members of the superfamily.

GDF-10 was identified from a mixture of PCR products obtained with the primers:

```
NSC1: 5′-CCGGAATTCAA(G/A)GT(G/A/T/C)GA(T/C)TT(T/C)GC(G/A/T/C)GA
                  (T/C)AT(A/C/T)GG(G/A,T/C)TGG-3′
NSC2: 5′-CCGGAATTC(A/G)CA(G/A/T/C)GC(A/G)CA(G/A)CT(T/C)TC(G/A/T/C)
                  AC(G/A/T/C)GTCAT-3′
NSC3: 5′-CCGGAATTC(A/G)CA(G/A/T/C)GC(A/G)CA(G/A/T/C)GA(T/C)TC
                  (G/A/T/C)AC(G/A/T/C)GTCAT-3′
```

PCR using primers NSC1 with NSC2 or NSC1 with NSC3 was carried out with cDNA prepared from 0.25 μg of lung or brain mRNA for 35 cycles at 94° C. for 1 min, 50° C. for 2 min, and 72° C. for 2 min. PCR products of approximately 300 base pairs were digested with Eco RI, gel purified, and subcloned in the Bluescript vector (Stratagene, San Diego, Calif.). DNA was prepared from bacterial colonies carrying individual subclones and sequenced. Of 11 clones that were sequenced, 9 corresponded to BMP-3, and two represented a novel sequence, which was designated GDF-10.

### EXAMPLE 2

### Expression Pattern and Sequence of GDF-10

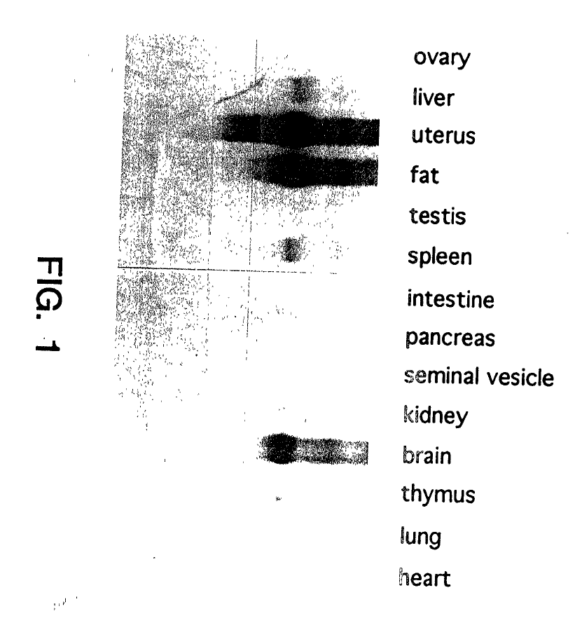

To determine the expression pattern of GDF-10, RNA samples prepared from a variety of adult tissues were screened by Northern analysis. 2.5 micrograms of twice polyA-selected RNA prepared from each tissue were electrophoresed on formaldehyde gels, blotted and probed with GDF-10. As shown in FIG. 1, the GDF-10 probe detected an mRNA expressed at highest levels in uterus, fat, and brain.

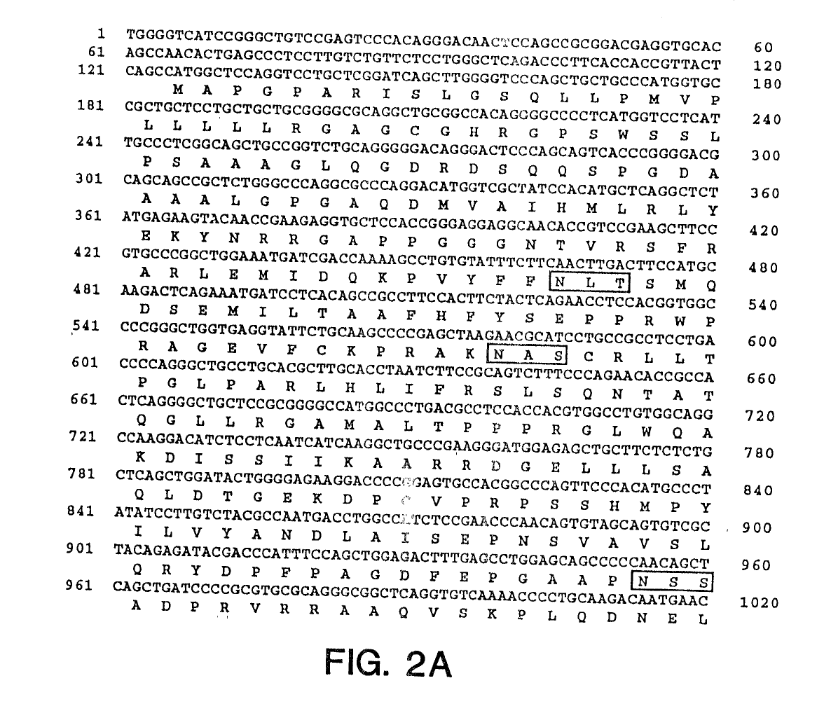

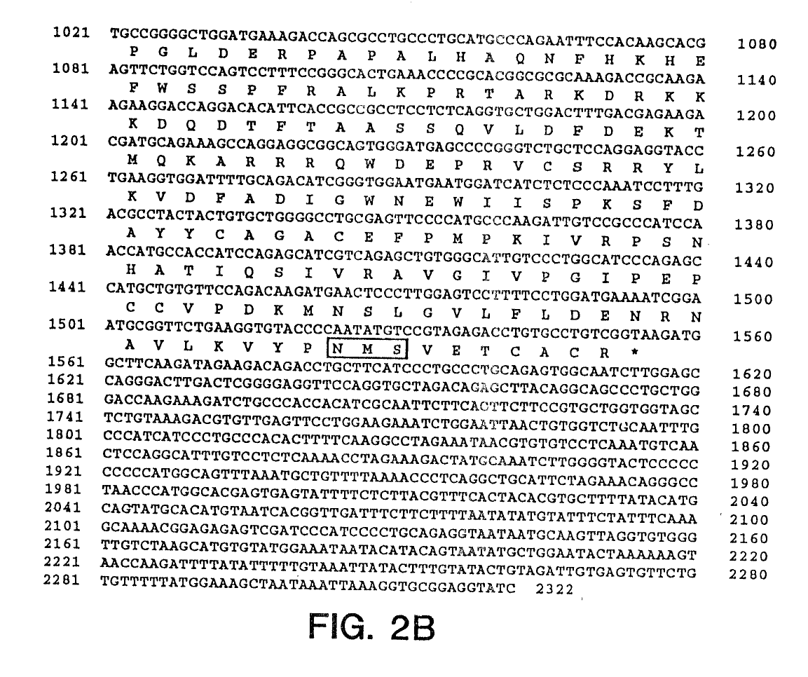

A murine uterus cDNA library consisting of 3×10⁶ recombinant phage was constructed in lambda ZAP II and screened with a probe derived from the GDF-10 PCR product. The entire nucleotide sequence of the longest of 7 hybridizing clones is shown in FIG. 2. Consensus N-glycosylation signals are denoted by plain boxes. Numbers indicate nucleotide position relative to the 5′ end. The 2322 bp sequence contains a long open reading frame beginning with a methionine codon at nucleotide 126 and potentially encoding a protein 476 amino acids in length with a molecular weight of 52.5 kD. The predicted GDF-10 amino acid sequence contains a hydrophobic N-terminal region, suggestive of a signal sequence for secretion, four potential N-linked glycosylation sites at asparagine residues 114, 152, 277, and 467 and a putative proteolytic processing site at amino acid 365. Cleavage of the GDF-10 precursor at this site would generate a mature GDF-10 protein 111 amino acids in length with a predicted unglycosylated molecular weight of 12.6 kD.

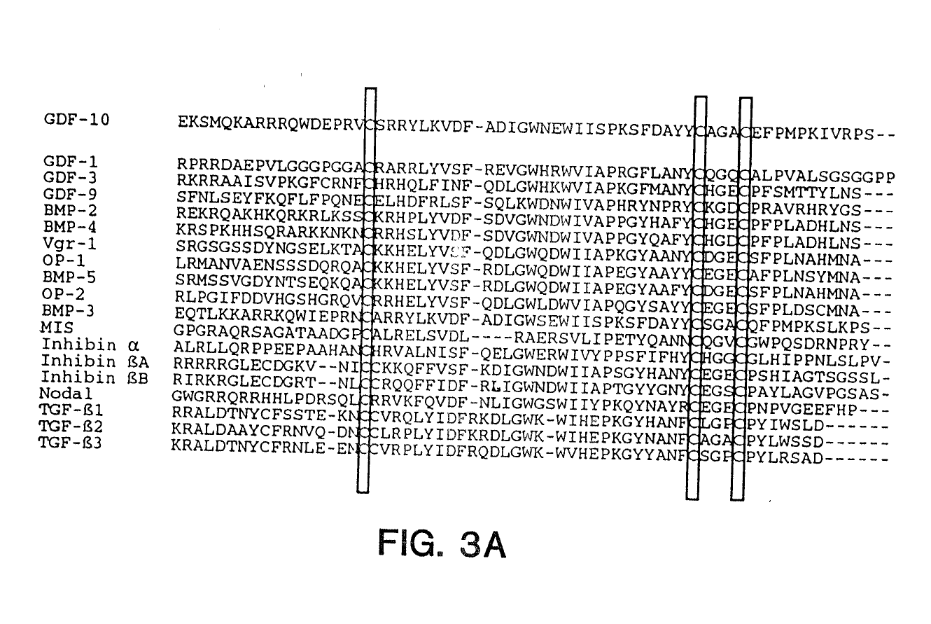

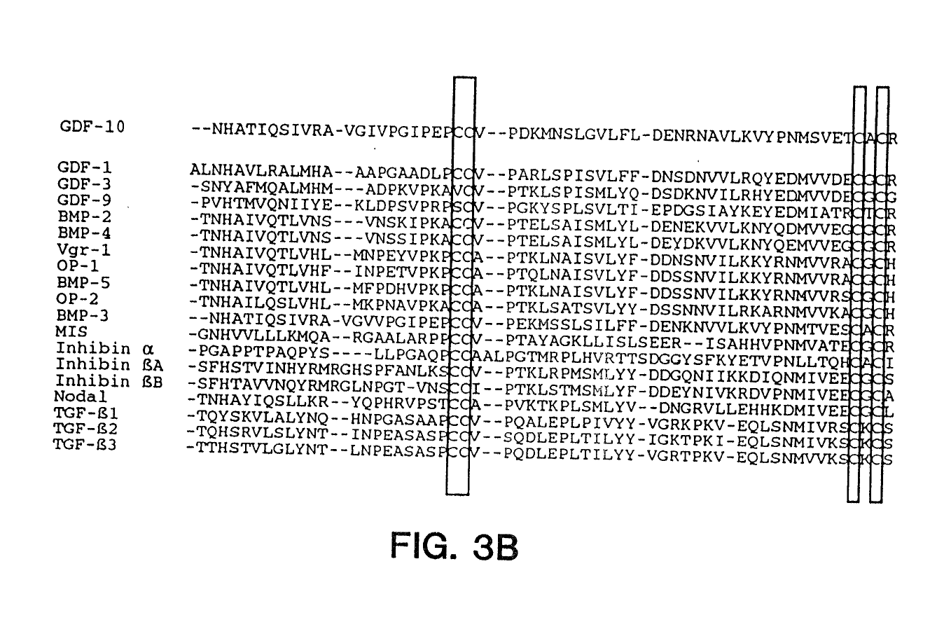

The C-terminal region of GDF-10 following the putative proteolytic processing site shows significant homology to the known members of the TGF-β superfamily (FIG. 3). FIG. 3 shows the alignment of the C-terminal sequences of GDF-10 with the corresponding regions of human GDF-1 (Lee, Proc. Natl. Acad. Sci. USA, 88:4250-4254, 1991), murine GDF-3 and GDF-9 (McPherron and Lee, J. Biol. Chem. 268:3444, 1993), human BMP-2 and 4 (Wozney, et al., Science, 242:1528-1534, 1988), human Vgr-1 (Celeste, et al., Proc. Natl. Acad. Sci. USA, 87:9843-9847, 1990), human OP-1 (Ozkaynak, et al., EMBO J., 9:2085-2093, 1990), human BMP-5 (Celeste, et al., Proc. Natl. Acad. Sci. USA, 87:9843-9847, 1990), human OP-2 (Ozkaynak, et al., J. Biol. Chem., 267:25220-25227, 1992), human BMP-3 (Wozney, et al., Science, 242:1528-1534, 1988), human MIS (Cate, et al., Cell, 45:685-698, 1986), human inhibin alpha, βA, and βB (Mason, et al., Biochem, Biophys. Res. Commun., 135:957-964, 1986), murine nodal (Zhou, et al., Nature, 361:543-547, 1993), human TGF-β1 (Derynck, et al., Nature, 316:701-705, 1985), humanTGF-β2 (deMartin, et al., EMBO J., 6:3673-3677, 1987), and human TGF-β3 (ten Dijke, et al., Proc. Natl. Acad. Sci. USA 85:47154719,1988). The conserved cysteine residues are boxed. Dashes denote gaps introduced in order to maximize the alignment.

GDF-10 contains most of the residues that are highly conserved in other family members, including the seven cysteine residues with their characteristic spacing.

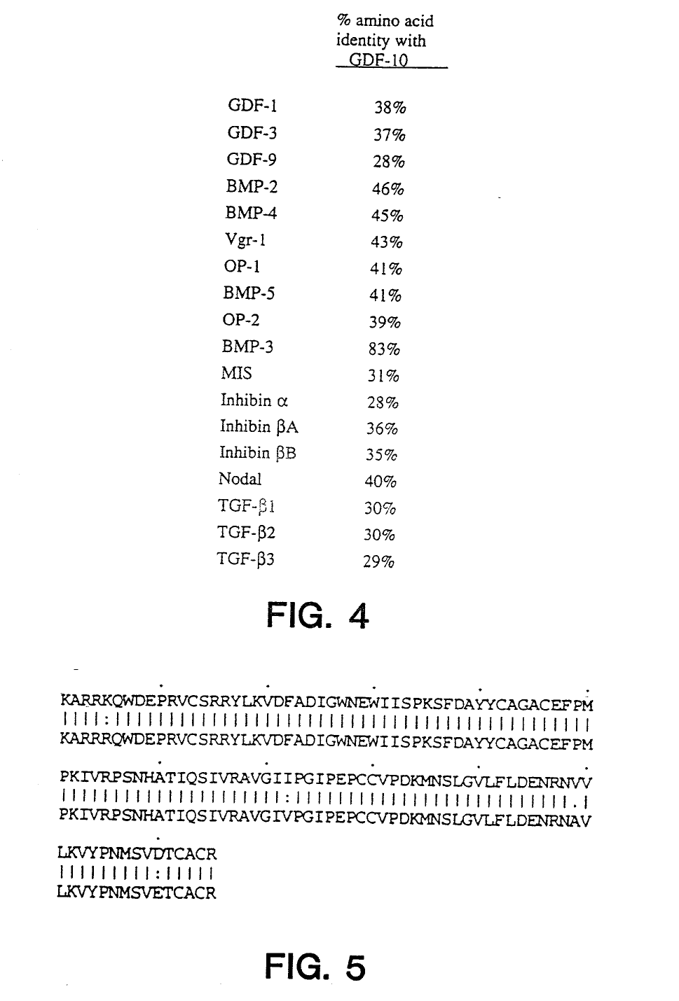

FIG. 4 shows the amino acid homologies among the different members of the TGF-β superfamily. Numbers represent percent amino acid identities calculated from the first conserved cysteine to the C-terminus. In this region, GDF-10 is most homologous to BMP-3 (83% sequence identity).

### EXAMPLE 3

### Isolation of Human GDF-10

To isolate human GDF-10, a human uterus cDNA library consisting of 16.2×10⁶ recombinant phage was constructed in lambda ZAP II and screened with a murine GDF-10 probe. From this library, 20 hybridizing clones were isolated. Partial nucleotide sequence analysis of the longest clone showed that human and murine GDF-10 are highly homologous; the predicted amino acid sequences are 97% identical beginning with the first conserved cysteine residue following the predicted cleavage site (FIG. 5).

### EXAMPLE 4

### Secretion of GDF-10 by Mammalian Cells

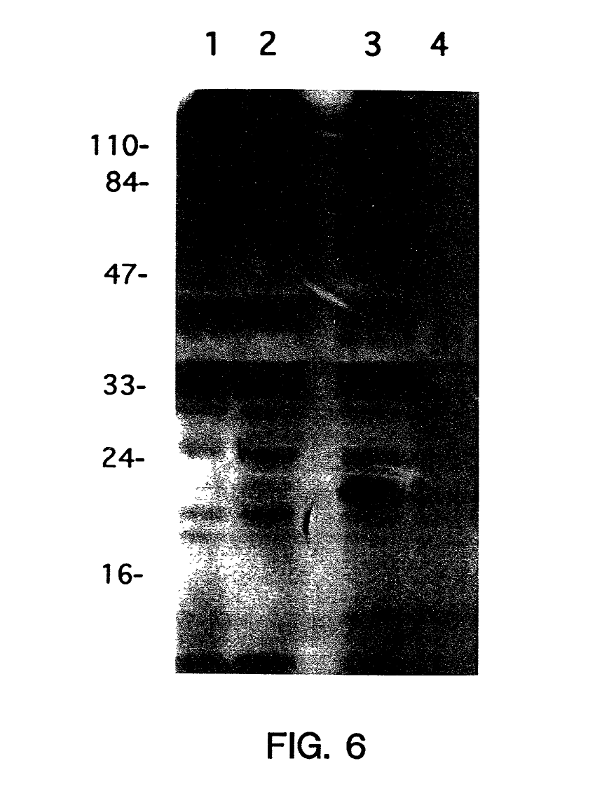

To determine whether GDF-10 is secreted by mammalian cells, the GDF-10 cDNA was cloned into the pcDNAI expression vector and transfected into 293 cells. Following DNA transfection, the cells were metabolically labeled with a mixture of [³⁵S]-cysteine and [³⁵S]-methionine, and labeled secreted proteins were analyzed by SDS-polyacrylamide gel electrophoresis. As shown in FIG. 6, additional bands were detected in cells transfected with a sense GDF-10 construct compared to an antisense control construct. The presence of multiple protein species most likely indicates that 293 cells are capable of proteolytically processing GDF-10. Hence, these data suggest that GDF-10 is secreted by these cells and that GDF-10 is cleaved, as predicted from the cDNA sequence.

Although the invention has been described with reference to the presently preferred embodiment, it should be understood that various modifications can be made without departing from the spirit of the invention. Accordingly, the invention is limited only by the following claims.

We claim:

1. A purified antibody that binds to growth differentiation factor-10 (GDF-10) or to immuogenic fragments thereof.

2. The antibody of claim 1, wherein the antibody is polyclonal.

3. The antibody of claim 1, wherein the antibody is monoclonal.

4. The antibody of claim 1, wherein the antibody includes Fab, Fv or other functional antibody fragments.
